(19) United States
(12) Patent Application Publication
(10) Pub. No.: US 2004/0247710 A1
(43) Pub. Date: Dec. 9, 2004

(54) Aromatherapy composition of essential oils for the relief of hormonal imbalances in women

(76) Inventor: Deborah Long Chambers, Irving, TX (US)

Correspondence Address: Deborah Long Chambers, 815 Ventura Park, Irving, TX, 75061, US

(21) Appl. No.: 10/455,331

(22) Filed: Jun. 6, 2003

(51) Int. Cl. A61K 35/78

(52) U.S. Cl. 424/745; 424/778; 424/770

(57) ABSTRACT

This invention is directed to an aromatherapy compound that relieves symptoms of female hormonal imbalances. It uniquely comprises an externally applied compound consisting of “essential oils” (highly concentrated oils extracted from plant cells for use in natural, therapeutic applications) that aid in the relief or reduction of symptoms of pre, post and peri-menopause and premenstral syndrome. The compound preferably comprises specific proportions of essential oil of geranium, essential oil of lavender, and essential oil of cypress. The preferred embodiment is to add the compound to a “carrier” (a base for diluting the aromatherapy compound) such as water. The preferred formula is approximately one part lavender, two parts geranium, and four parts cypress blended in approximately four hundred, twenty-five parts of the carrier.

### BACKGROUND OF THE INVENTION

1. Field of the Invention

The present invention relates to an essential oil composition and, more particularly, to an essential oil composition, which can relieve the symptoms, caused by pre, post and menopausal hormonal imbalances in women.

Essential oils have been used for thousands of years in aromatherapy. The ancient Chinese are generally acknowledged as the founders of aromatherapy, but it is more than likely that quite early in the history of civilisation man had realised that certain aromatic plants could help restore his health. Aromatic substances were also used by the ancient Egyptians and Ancient Greeks as medicinal perfumes.

In the 10th century the Arabs were extracting essential oils from aromatic plants and using them medicinally. The Knights of the Crusades brought aromatic essences and waters back to Europe from the Middle East and they became so popular that perfume began to be manufactured and was well established by the end of the 12th century. The importance of aromatic plants for other purposes was realised early. When the bubonic plague reached England around the middle of the 14th century, fires were ordered in the streets at night, burning aromatic frankincense and pine; indoors, incense and perfumed candles were burnt to combat infection and disguise the stench of death; pomanders made from aromatic gums and resins were worn on ribbons round the neck to protect the wearers from the dreaded Black Death.

By the turn of the 18th century essential oils were widely used in medicinal preparations and Salmon's dispensary of 1896 contains recipes for numerous aromatic remedies. In the 19th century, essential oils were subjected to more scientific investigation, and it was discovered that some of them could be synthesized from other materials. As it is always quicker and cheaper to produce the laboratory versions than natural plant extracts, true essential oils began to fall from demand. Today, many of our medicines and perfumes contain so-called essential oils, though often they are mere imitations; while synthetics may smell like the real thing, they do no possess the same therapeutic properties.

Essential oils may be extracted from plants in a number of ways. One of the oldest methods is distillation, practiced in ancient Persia, Turkey and India thousands of years ago. The Egyptians were preparing essence of cedar woods for embalming and other purposes around 2000 BC; the wood was heated in a clay vessel covered by a screen of wool fibers through which the steam had to pass. The essence was obtained by squeezing out the impregnated wool. The Arabs are credited with having popularized distillation in the late 10th century. They began with extract of rose petals then experimented with other aromatic materials. Today, distillation remains the most commonly used means of extracting essential oils.

Other methods include enfleurage, often used for delicate petals like jasmine and tuberose; maceration, for tougher flowers and leaves, roots and bark; solvent extraction, the preferred method for gums and resins like myrrh and galbanum; and hand expression, chiefly employed for squeezing the highly aromatic oils from thick-skinned citrus fruit like oranges, tangerines and lemons.

2. Discussion of the Prior Art

The prior art is replete with compositions and compounds that teach of medicinal and cosmetic uses.

The U.S. patent to Manella (U.S. Pat. No. 6,322,823), issued Nov. 27, 2001, discloses an aromatherapy compound for the relief of symptoms of premenstrual syndrome. This patent teaches the use of an externally applied compound comprised of equal proportions of the essential oils of geranium (1 milliliter), clary sage (1 milliliter) and orange (1 milliliter), blended in 120 milliliters of a carrier.

The U.S. patent to Morrow (U.S. Pat. No. 5,707,630), issued Jan. 13, 1998, teaches of an herbal compound for the relief of premenstrual syndrome and menopausal symptoms. The disclosed herbal compound comprises red raspberry, bayberry, blue cohosh, capsicum, cascara sagrada, damania, ginger, valerian and a binding agent, such as cellulose, to hold the herbal components together. This compound, and the binder, is formed into a tablet and ingested.

Newmark et al. (U.S. Pat. No. 6,242,012) received a patent on Jun. 5, 2001, for teaching of an herbal composition for promoting hormonal balance in women. This composition contains supercritical extracts of ginger, rosemary and evening primrose oil and either supercritical or regular extracts of black cohosh, dong quai, schizandra berry, chaste tree berry and rosemary.

Fletcher et al. (U.S. Pat. No. 6,280,751) issued on Aug. 28, 2001, and teaches of an essential oil composition. The patent discloses the use of an essential oil composition in conjunction with herbs and/or spices. The thrust of Fletcher is directed toward this unique combination.

None of these patents either teaches or suggests the use of a combination of the essential oils of geranium, lavender and cypress, in particular proportions, and in a carrier, such as water. As will be seen in greater detail hereinafter, the present invention involves a unique and specific blend of chosen functional ingredients that can be delivered as a misting spray, for alleviating symptoms caused by hormonal imbalances in women.

### SUMMARY OF THE INVENTION

This invention relates to herbal compositions. More particularly, this invention relates to an herbal composition that can alleviate pre, post, and menopausal symptoms. The present invention features an aromatherapy composition comprised of three essential oils for promoting hormonal imbalances in women.

From the commencement of menstruation, until the termination of menopause, the female body can experience wide fluctuations in baseline hormonal levels. When such fluctuations cause an imbalance in the hormonal composition, certain chemical reactions occur which cause what are commonly referred to as PMS and/or menopausal symptoms. These symptoms include headaches, cramping, nausea, inflammation, increased agitation, anxiety, tension, restlessness, decreased digestive tract activity, depression, moodiness and severe mood swings. Hormone Replacement Therapy and conventional treatments for hormonal imbalance conditions like PMS in women are often the topic of controversy. Few would question that the treatment protocols are fraught with acute side effects and potential long-term health concerns.

There is an apparent need for safe and effective hormonal health/balance programs with a basis in modem science for women today. According to a growing body of research, the eicosanoid cascade plays a critical role in women's health and hormonal balance. An imbalance correlates not only with uncomfortable menstrual cycles, resulting in conditions like PMS, but also might relate to serious degenerative conditions like osteoporosis, heart disease and cancer. Historical and scientific research confirms that aromatherapeutic alternatives exist which can safely bring more comfort and balance to women's hormonal cycles.

Accordingly, a primary object of this invention is to provide a composition of essential oils providing aromatherapy benefits.

It is therefore an object of the invention to provide a composition of essential oils that alleviates symptoms caused by hormonal imbalances in women.

It is another object of the invention to provide an aromatherapeutic compound that can be administered as a mist with positive physiological and psychological affects.

It is also an object of the invention to provide an essential oil compound using geranium, lavender and cypress for aromatherapy.

It is a further object of the invention to provide a composition of essential oils providing cosmetic benefits.

These and other objects, features and advantages will be more apparent from a study of the enclosed text.

It is also an object of the invention to provide a composition of essential oils that relieve symptoms such as: hot flashes, night sweats, stress, nervous tension, swelling, and overheating (e.g. due to physical exertion).

### DESCRIPTION OF THE PREFERRED EMBODIMENT

This disclosure of the invention is submitted in furtherance of the constitutional purposes of the U.S. Patent Laws “to promote the progress of science and useful arts” (Article 1, Section 8).

Generally speaking this invention relates to an essential oil compound that aids in the relief of symptoms caused by female hormone imbalances. This invention is directed to an aromatherapy compound using a unique combination of natural oils extracted from plant cells to combat hormonal imbalance symptoms.

The female body, from the commencement of menstruation until the termination of menopause, can experience wide fluctuations in baseline hormonal levels. When such fluctuations cause an imbalance in the hormonal composition, certain chemical reactions thereto present as symptoms which include headaches, cramping, nausea, inflammation, increased agitation, anxiety, tension, restlessness, decreased digestive tract activity, depression, moodiness and severe mood swings.

These symptoms, commonly referred to as PMS and/or menopausal symptoms, can be relieved. For example, additional hormones can be ingested to compensate for the specific hormones in which the body is deficient. Alternatively, substances can be introduced which offset and/or neutralize the chemical reactions that gave rise to the symptoms. Given the changing characteristics of the hormonal imbalance during the menstrual cycle and/or menopause, as well as the variations in the specific hormonal imbalance from person to person, it would be advantageous to introduce a compound which adapts to meet the specific needs of each person, both over time as well as from person to person.

Essential oils possess numerous properties that make them useful for treating many of our most common health and beauty troubles. Essential oils are aromatic droplets found in minute quantities in the flowers, stems, and leaves, roots and barks of aromatic plants. They are not true oils in the manner of lubricant vegetable oils, but highly fluid and exceptionally volatile. For the purposes of this invention, an essential oil is a predominately volatile material or materials isolated by some physical (as opposed to chemical) process from an aromatic, single species, and botanical source.

The most widely used process for the isolation of essential oils is steam distillation of plant matter, although dry distillation and solvent extraction are also used. A botanical source is aromatic if any animal, not just a human, can detect an odor; aromatic thus, is simply an indication that some volatile component is present in the plant. The oils extracted by the physical process can contain some non-volatile material, as is well known in the art. Essential oils have been known for centuries in many cases and even millennia, and this term is well known in the art.

Essential oils are complex mixtures of different organic molecules-terpenes, alcohols, esters, aldehydes, ketones and phenols. Synthetic oils are usually made from one or more of the constituents predominant within a particular essential oil; menthol, for example, often substitutes for mint and eucalyptol for eucalyptus. However, there are sound reasons for believing that it is the interaction between each and every component that gives an essential oil its particular character and unique therapeutic properties.

The inventive compound preferably comprises specific proportions of essential oil of geranium, essential oil of lavender, and essential oil of cypress. The preferred formula is approximately 1 part lavender, 2 parts geranium, and 4 parts cypress blended in approximately four hundred, twenty-five parts of the carrier. In the preferred embodiment, a “carrier” (a base for diluting the aromatherapy compound) is added to the compound, such as water. Other known carriers include vegetable oil (such as sweet almond oil, etc.) Specifically, the preferred embodiment comprises the formula of 100 ml of water (carrier), 0.94 ml of cypress, 0.47 ml of geranium and 0.235 ml of lavender.

The inventive composition uses the unique combination of natural, extracted essential oils to combat symptoms of:

1) hot flashes;

2) night sweats;

3) stress;

4) nervous tension;

5) swelling; and

6) over heating (e.g. due to physical exertion).

Reactivity to stress varies with the individual. Some individuals thrive on stress whereas in other individuals, the same stress drives them towards illness. Researchers are discovering that stress decreases productivity, and eventually often leads to illnesses. This reactivity to stress can be brought about by the daily hassles—the repeated or chronic strains of everyday life. Stress may be defined as including: decrease in degree of relaxation; decrease in happiness; decrease in calmness; increase in fear; increase in tension; increase in emotional sensitivity; increase in anger; and/or increase in anxiety. The use of chemical agents, such as essential oils in the instant, is employed to modify the effects of stress, tension, and anxiety.

The essential oil of geranium (Pelargonium Odorantissimum) is distilled from the leaves of the plant. Its principal constituents include geraniol, citronellal, linalol, terpineol and alcohols. Essential oil of geranium is an adrenal cortex stimulant and has a balancing effect on the body. Since hormones secreted by the adrenal cortex are primarily regulators of hormones secreted by other organs, including sex hormones, it is extremely effective in conditions where hormones fluctuate, as in the female menstrual cycle and menopause. Essential oil of geranium is a natural diuretic, which is particularly helpful in eliminating excess fluid retention commonly associated with female hormone imbalances. It also has antidepressant and calming qualities, which may assist in easing mood swings.

The essential oil of Lavender (Lavandula angustifolia) helps to promote mental clarity and it helps to eliminate lack of concentration, fear, headaches, and aids in improving ailments of the eye. Lavender has a high ester content that gives it a calming, almost sedative effect. It also has a slight analgesic effect. It also stimulates circulation and has anti-inflammatory and antiseptic properties.

The essential oil of Cypress (Cupressus sempervirens L.) is extracted by steam distillation from the needles and twigs. Its main constituents are Pinene and camphene among others. It is pale yellow to greenish-olive liquid with a smoky, sweet-balsamic odor. It is classified as antirheumatic, and antiseptic, antispasmodic (e.g. for muscle tension relief, hot flash vascular shifting, nervous tension, as well as itching). It is further classified as astringent (e.g. for skin and facial hair), diuretic (e.g. for swelling and fluid retention), hepatic, styptic (i.e., stops bleeding), tonic, and vasoconstrictive (e.g. for varicose veins, hemorrhoids).

It was highly valued as a medicine and as incense by ancient civilizations. It is still used as incense for purification by the Tibetans. It is useful where there is excessive loss of fluid, such as heavy perspiration, or menstrual loss and diarrhea. It is non-toxic, non-irritant, and non-sensitizing.

Sprayed into the air near the face of a user, this essential oil composition has immediate and long-lasting effects in relieving the symptomatic relief of ailments caused by hormonal imbalances. The reasons for these reactions are that odor molecules are perceived by thousands of tiny nerve cells in the nose and that each of these nerves is connected to that part of the brain that is concerned with emotional drives, creativity and sexual behavior. It is known that pure essential oils in aromatherapy blends have a positive influence on the psyche. Unlike a drug that is ingested orally or injected subcutaneously, the substances used for the practice of the present invention are inhaled and/or absorbed by means of transdermal penetration, i.e., transdermally absorbed into the bloodstream.

Since other modifications and changes varied to fit a particular operating requirements and environment will be apparent to those skilled in the art, the invention is not considered limited to the example chosen for purposes of disclosure, and covers all changes and modifications which do not constitute a departure from the true spirit and scope of the invention. For example, the essential oils used in the present invention may be used with other carriers, such as a cream constitute, and applied to the surface of parts of the body. In such embodiments, additional essential oils may be compounded for further medicinal benefits.

### COMMENTARY

Modern medicine and much of aromatherapy alike are largely devoted to healing of the sick. The practice of this invention is directed to alleviation of physiological and/or subjective stress-induced reactivity in persons of a level not requiring the attention of the medical practitioners. Usually the reactivity to an ordinary stress situation (reactivity being measurable by a pulse-like elevation in systolic blood pressure) will not be accompanied by any untoward observable reaction. As a result, although substances employed in practice of this invention are known to have physiologic activity, they are suggested in alternative medicine and aromatherapy within a different use context than practice of this invention.

The results obtained through practice of this invention are comparable to results obtainable from meditation and biofeedback, including, notably, damping of the systolic blood pressure surges arising from stressful situations. However, unlike meditation or biofeedback, no training period is required In the use of the stress reactivity-reducing substances of our invention. Unlike anti-anxiety drugs, the effect of the volatile compositions of matter employed in the practice of our invention, (e.g., lavender essential oil, cypress essential oil, and/or geranium essential oil) is prophylactic in nature, reducing (the physiological and/or subjective) reactivity to stress when stress conditions exist.

Having thus described the invention, what is desired to be protected by Letters Patent is presented in the subsequent appended claims.

What is claimed is:

1. A medicinal composition for transdermal administration for the symptomatic relief of hormonal imbalances in women, said composition comprising:
the essential oils of cypress, geranium and lavender; and a carrier.

2. The medicinal composition as in claim 1, wherein,
said essential oils are in a specific ratio to one another, said essential oil ratios consisting of:
four parts cypress,
two parts geranium, and
one part lavender.

3. The medicinal composition as in claim 2 wherein,
said carrier is water.

4. The medicinal composition as in claim 3 wherein,
said carrier of water is at a specified ratio of four hundred, twenty-five parts, relative to each of the essential oils.

5. An aromatherapy composition for transdermal administration via a misting spray, said composition aiding in the relief of the symptoms of hot flashes; night sweats; stress; nervous tension; swelling; and over heating (e.g. due to physical exertion) said composition consisting of:
the essential oil of cypress,
the essential oil of geranium,
the essential oil of lavender, and
a carrier.

6. The aromatherapy composition as in claim 5 wherein,
the essential oil of cypress is present in the amount of approximately 0.94 ml.

7. The aromatherapy composition as in claim 6 wherein,
the essential oil of geranium is present in the amount of approximately 0.47 ml.

8. The aromatherapy composition as in claim 7 wherein,
the essential oil of lavender is present in the amount of approximately 0.235 ml.

9. The aromatherapy composition as in claim 8 wherein,
the carrier is water, and is present in the amount of approximately 100 ml.

10. A method of preparing a medicinal, aromatherapeutic composition that provides symptomatic relief associated with female hormonal imbalances, said method steps comprising:
providing a container for housing said composition,
providing approximately 0.94 ml of the essential oil of cypress,
providing approximately 0.47 ml of the essential oil of geranium,
providing approximately 0.235 ml of the essential oil of lavender, and
mixing said essential oils.

11. The method of claim 10, further comprising the steps of providing a carrier of approximately 100 ml of water to said mixture of essential oils.

12. The method of claim 11 wherein said providing a container step further comprises:
providing a container having a spray dispensing mechanism for the administration of said medicinal composition transdermally as a spray;
and insulating means for reducing thermodynamic, and photodynamic reduction transfers of the composition.
